(19) United States
(12) Patent Application Publication
(10) Pub. No.: US 2019/0083057 A1
(43) Pub. Date: Mar. 21, 2019

(54) ULTRASONIC CONTROL FOR INTRAVASCULAR TISSUE DISRUPTION

(71) Applicant: SHIFAMED HOLDINGS, LLC, Campbell, CA (US)

(72) Inventors: Tom SAUL, Moss Beach, CA (US); Amr SALAHIEH, Saratoga, CA (US)

(21) Appl. No.: 16/078,942

(22) PCT Filed: Feb. 24, 2017
(86) PCT No.: PCT/US2017/019426

§ 371 Date: Aug. 22, 2018

Related U.S. Application Data
(60) Provisional application No. 62/300,678, filed on Feb. 26, 2016.

(51) Int. Cl. A61B 8/12; A61B 8/08; A61M 5/145; A61M 5/00

(52) U.S. Cl. CPC A61B 8/12; A61B 8/0858; A61M 5/14526; A61B 8/445; A61M 5/007; A61B 8/481; A61B 8/0891; A61M 5/1454

(57) ABSTRACT

Medical systems and devices adapted to deliver a fluid agent to target tissue within a patient.

### CROSS REFERENCE TO RELATED APPLICATIONS

This application claims the benefit of U.S. Provisional Application No. 62/300,678, filed Feb. 26, 2016, which is incorporated by reference herein.

This application also incorporates by reference herein the following applications: U.S. application Ser. No. 13/071,436, filed Mar. 24, 2011, U.S. Prov. App. No. 61/317,231, filed Mar. 24, 2010; U.S. Prov. App. No. 61/324,461, filed Apr. 15, 2010; U.S. Prov. App. No. 61/589,669, filed Jan. 23, 2012; and U.S. Prov. App. No. 61/642,695, filed May 4, 2012.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Medical fluid delivery systems have been described that can deliver fluid to a target location within a patient. In some applications a fluid source houses a fluid that is delivered from the fluid source through a delivery device positioned in the patient and into the patient. Needleless applications include a delivery device that has an aperture therein, and fluid is allowed to be moved from the fluid source, through the delivery device, out of the aperture, and into the patient.

Some applications attempt to generate a transient relatively high fluid pressure at a location along the fluid path in an effort to deliver the fluid into the patient at a relatively high-velocity. U.S. Pat. No. 6,964,649, for example, describes a fluid source that is capable of generating a transient high-pressure to deliver fluid into tissue. Deficiencies of these and other previous attempts are set forth in more detail below.

### SUMMARY OF THE DISCLOSURE

One aspect of the disclosure is a method of delivering fluid into a patient, comprising: maintaining a fluid agent under a substantially constant high-pressure within a fluid reservoir; opening a fluid control downstream of the fluid reservoir from a closed configuration to allow the fluid agent maintained at substantially constant high-pressure to flow under high-pressure from the fluid reservoir to a fluid aperture disposed downstream to the fluid control; and delivering the fluid agent at high-velocity out of the aperture and into the patient.

In some embodiments opening the fluid control downstream the fluid reservoir comprises opening a fluid control that is disposed external to the patient.

In some embodiments the method further comprises positioning a delivery device comprising the aperture within a renal artery, and wherein the delivering step comprises delivering the fluid agent at high-velocity out of the aperture and into the patient such that the fluid agent interacts with nerves surrounding the renal artery and disrupts neural communication along the nerves to reduce hypertension.

In some embodiments maintaining a fluid agent under substantially constant high-pressure comprises maintaining a fluid agent at between 750 psi and 5000 psi.

In some embodiments the method further comprises positioning a delivery device comprising the aperture within a lumen, and positioning the aperture such that it faces radially outward from the longitudinal axis of the delivery device. The method can also include expanding an expandable member to position the aperture into engagement with the lumen wall.

Expanding the expandable member can reconfigure a fluid delivery line secured to the expandable member.

In some embodiments the method further comprises closing the fluid control to thereby control the volume of the fluid agent that is delivered out of the fluid aperture.

In some embodiments delivering the fluid agent at high-velocity out of the aperture and into the patient comprises delivering the fluid agent at between 50 m/sec and 400 m/sec.

In some embodiments the fluid agent flows out of the fluid reservoir at between about 5 mL/min and about 40 mL/min.

In some embodiments delivering the fluid agent at high-velocity out of the aperture and into the patient comprises delivering the fluid agent in a fluid pulse with a duration of between about 50 and 500 msec.

In some embodiments delivering the fluid agent comprises delivering the fluid agent in a fluid pulse of between about 10 uL and about 500 uL of the fluid agent.

One aspect of the disclosure is an apparatus for delivering fluid to a target location within a patient's body, comprising: a high-pressure source adapted to maintain a fluid within a fluid reservoir at a substantially constant high-pressure; a fluid delivery device comprising a fluid delivery aperture, wherein the delivery device is adapted to be positioned within the patient; and a fluid control disposed downstream the high-pressure source and upstream the aperture, wherein the fluid control is configured to control the flow of fluid therethrough and to modify fluid communication between the fluid reservoir and the fluid delivery aperture.

In some embodiments the fluid control is a valve with an open configuration and a closed configuration.

In some embodiments the fluid control is adapted to be disposed external to the patient.

In some embodiments the apparatus further comprises an expandable member adapted to reposition the aperture against the lumen wall.

In some embodiments the fluid control is adapted to be activated from an off state to an on state and then back to the off state, with both on/off and off/on transitions less than about 15 msec.

In some embodiments the fluid delivery aperture has a diameter between about 1 mil and about 5 mils.

In some embodiments the high-pressure fluid source is adapted to maintain a fluid agent under pressure between 750 psi and 5000 psi within the fluid reservoir.

A visualizing high-pressure needleless injection system which comprises any or any combination of the following capabilities:

An imaging apparatus capable of differentiating tissues adjacent to and to a depth corresponding to the depth of penetration of a delivered injectate. The imaging apparatus used to determine the location of a target tissue bed, within surrounding tissues, into which an injectate is to be delivered. Embodiments of the imaging apparatus can comprise any combination of 2D and or 3D, with or without time. The imaging apparatus capable of assessing that an injectate is being and or has been delivered to the predetermined depth within and or to a target tissue.

A fluidics apparatus to deliver an injectate via a needleless means where the injectate can be expressed from an outflow orifice according to a predetermined pressure(time) waveform into a target tissue. Such an apparatus capable of delivering fluids at velocities greater than 50 m/sec and up to 400 m/sec. The delivery apparatus capable of volume metering and pressure control.

A delivery apparatus comprising a means of holding the outflow orifice adjacent a tissue surface in proximity to a target tissue.

A control system for controlling the pressure time waveform such that the pressure at the outflow orifice follows a predetermined pressure (time) waveform

A control system which uses the imaging information associated with the location or depth, of various tissue substrates in the field of view, as an input into a process for developing a pressure/time waveform output to be delivered to a control algorithm for controlling a high-pressure injection such that the injectate is delivered to the target tissue and or depth.

A control system which uses the imaging information associated with the depth of penetration of a delivered portion of injectate , and or the relative location of the delivered portion of injectate relative to a target tissue location as an input into a feedback loop to adjust the pressure(time) waveform at the exit aperture to adjust the depth of penetration of the injectate to improve the accuracy of the delivery of the injectate to a predetermined depth and or into the target tissue.

An injectate for use in such a visualizing high-pressure needleless injection system comprising a contrast agent. The contrast agent comprising any or any combination of the following, micro or nano particulate matter comprising an acoustic impedance less than 1 Rayl or greater than 2 Rayl, and more preferably less than 0.5 Rayl or greater than 4 Rayl. In some embodiments the particles may be Adsorbable such as collagen particles, in yet others the particles may additionally comprise some therapeutic agent or an ablating agent. When used the contrast agent may additionally be used to enhance the absorption of therapeutic ultrasound such that a non-ablating dose of energy will be rendered ablating in the location of the contrast agent. Alternatively, or in combination the injectate contrast may be enhanced by using an injectate over saturated with gas or gases as measured at body temperature and atmospheric pressure.

One aspect of the disclosure is a computer executable method adapted to control one or more fluid delivery parameters, comprising: receiving as input a signal from an ultrasound transducer optionally indicative of a tissue parameter, the transducer optionally carried by an elongate medical device, the elongate medical device optionally comprising a fluid delivery lumen and a small aperture at a distal region of the fluid delivery lumen, the small aperture defining the outflow for the fluid. The computer executable method can include selecting, based on an assessment of the received input (optionally indicative of a tissue parameter), one or more fluid delivery parameters for controlling the high velocity delivery of a fluid that is optionally to be delivered from a fluid reservoir to the small aperture and into a tissue.

### BRIEF DESCRIPTION OF THE DRAWINGS

FIG. 1 shows an exemplary fluid delivery system.

FIG. 2 depicts a portion of an exemplary fluid delivery system.

FIG. 3 illustrates an exemplary high-pressure fluid source.

FIG. 4 shows an exemplary breadboard fluid control system configured for a pump source described in FIG. 3.

FIG. 5 illustrates an exemplary embodiment of a high-velocity fluid delivery system adapted to deliver a fluid agent under high-pressure into a patient.

FIGS. 6 and 7 illustrate an exemplary high-pressure fluid source.

FIG. 8 is a graph illustrating pressure vs. time and illustrates the pressure of the fluid within the fluid reservoir 13 in FIGS. 6 and 7.

FIG. 9 illustrates an embodiment of a fluid delivery system in which an exemplary high-pressure fluid source is coupled to an elongate delivery device.

FIGS. 10 and 11 illustrate alternative embodiments of alternate metering outflow valve variations.

FIGS. 12 and 13 illustrate two variations that incorporate automatic high-pressure refilling systems.

FIGS. 14 and 15 illustrate exemplary distal regions of two exemplary delivery devices.

FIGS. 16A-16C illustrates an expandable member that is radially offset with respect to a catheter shaft.

FIG. 17 illustrates a typical pressure diameter profile associated with an artery.

FIG. 18 illustrates the pressure waveform generated in the system from FIG. 4.

FIGS. 19A-19D show various images of tissue treated with fluid injections exhibiting a pressure pulse similar to that illustrated in FIG. 18.

FIGS. 20A-20D illustrate different generalized waveforms useful in needle-less injection of fluid agents into periluminal spaces.

FIGS. 21A and 21B are fluoroscopic images illustrating a cloud of injectate.

FIGS. 22A and 22B illustrate features of a distal end of a high-velocity fluid delivery system comprising an ultrasonic transducer.

FIG. 23 illustrates a cross section features of a distal end of a high-velocity fluid delivery system comprising an ultrasonic transducer placed within a vessel and possible signals captured by the ultrasonic transducer.

FIGS. 24A-24C illustrate a selection of possible high-velocity fluid delivery scenarios.

FIG. 25 is a block diagram of a possible image controlled high-velocity fluid delivery procedure.

FIG. 26 illustrates a high-velocity fluid delivery source.

### DETAILED DESCRIPTION

The disclosure herein relates generally to medical devices, and particularly to systems and methods of use for delivering a fluid agent to a target location within a patient. In some embodiments the devices and systems herein are used to deliver a fluid agent out of an aperture in a delivery device, through tissue adjacent the aperture (which may be referred to herein as “intermediate tissue”), and to target tissue that is more distant from the aperture than the tissue adjacent the aperture (which may be referred to herein as “target tissue”). Exposing the target tissue to the fluid agent causes a desired change in the target tissue.

In some embodiments it is desirous to cause minimal damage to the intermediate tissue while delivering the fluid agent to the target tissue. Minimal damage to the intermediate tissue is generally considered similar or less than is caused by a small gauge needle penetrating the intermediate tissue, and substantially less than is caused to the intermediate tissue by the delivery of RF ablation energy delivered at the lumen wall for treatment of a tissue peripheral or distant to the lumen wall. If RF energy is delivered the lumen wall will sustain more damage than the target tissue because the RF energy source is adjacent to the lumen wall and the energy density at the lumen wall is greater than at the target tissue. As described herein the fluid agent pierces through, or penetrates through, the intermediate tissue with minimal damage to the intermediate tissue. One manner in which the damage is minimized is by delivering a high-velocity fluid jet out of the aperture. The disclosure herein focuses primarily on creating the high-velocity fluid jet by creating a relatively high-pressure gradient across a relatively small fluid aperture. The high-velocity fluid delivery also ensures that minimal leaking of the fluid agent into the lumen occurs when the fluid agent is delivered out of the aperture.

The one or more apertures can be positioned in any lumen within the body, and as used herein “lumen” includes spaces in the body other than tubular structures. For example without limitation, any portion of the vasculature, the interior of the gastrointestinal tract, the esophagus, urethra, and the stomach are “lumens” as used herein.

In some embodiments the intermediate and target tissues are characterized as the same type of tissue, but the target type of tissue is more distant, relative to the aperture, than the intermediate type of tissue. In some embodiments the intermediate and target tissues are different types of tissue.

An exemplary situation in which it may be desirable to minimize damage to the intermediate tissue is when the fluid is being delivered through the lumen of an arterial wall to target tissue peripheral to the lumen wall. For example, as descried herein, in some uses the fluid is delivered at high-velocity through a renal artery lumen and wherein the target tissue is the medial layer and/or adventitial layers, in which nerves that innervate the kidneys are disposed. In some methods of use it is desirable to deliver a fluid agent to the medial and/or adventitial layers to disrupt the neural tissue, while minimizing the damage to the renal artery lumen wall.

The systems herein include a fluid reservoir adapted to house a fluid agent therein. The systems also include a delivery device with at least one aperture adapted to allow for the delivery of the fluid agent from the reservoir and out of the aperture and into the patient at high-velocity. The velocity of the fluid exiting the aperture is related to the pressure gradient of the fluid agent across the aperture, among other variables. Some previous approaches have attempted to generate a high transient fluid pressure at a fluid reservoir disposed external to a patient in order to generate a high-velocity fluid delivery within the patient. In embodiments herein, however, the systems and methods of use generate the high-velocity fluid delivery into the patient by maintaining the fluid in the fluid reservoir at a high-pressure. While the fluid agent is being maintained under high-pressure in the fluid reservoir, a fluid control distal, or downstream to, the fluid reservoir is opened, which delivers the fluid agent under high-pressure out of the fluid reservoir, towards the aperture, and out of the aperture at a high-velocity.

FIG. 1 illustrates conceptually an exemplary fluid delivery system 102 that includes high-pressure fluid source 104 that is adapted to maintain a fluid agent under high-pressure, a high-pressure fluid control, and fluid delivery device 106 capable of communication with high-pressure fluid source 104. High-pressure fluid source 104 includes at least one fluid reservoir adapted to house a fluid agent therein. Delivery device 106 includes at least one fluid delivery lumen adapted to receive fluid from the fluid reservoir, and at least one aperture, or port, adapted to allow the fluid agent to be delivered into the patient from delivery device 106.

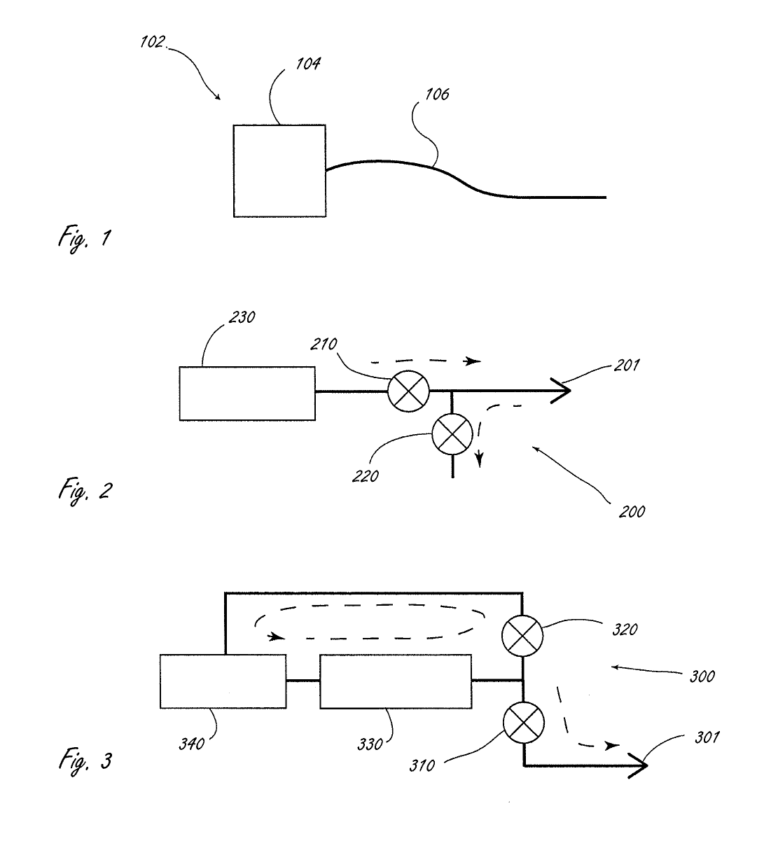

FIG. 2 depicts a portion of an exemplary fluid delivery system illustrating fluid reservoir 230 adapted to house a fluid agent therein, inline fluid control 210, and optional bypass fluid control 220. Fluid controls 210 and 220 can be any type of suitable valve. Fluid control 210 is disposed between delivery device inflow 201 and the fluid reservoir 230. Bypass fluid control 220 “T's” off the outflow line and empties to a low-pressure exhaust point such as ambient pressure. During idle, fluid control 210 is in a closed configuration and fluid control 220 is in an open configuration. In idle, also referred to herein as the primed state, the fluid in fluid reservoir 230 is maintained under substantially constant high-pressure. When fluid is to be delivered from the reservoir 230 under high-pressure, fluid control 220 is closed, and fluid control 210 is then opened for the requisite period of time to cause the fluid to be delivered under high-pressure out of the reservoir. Fluid control 210 is then closed and fluid control 220 is opened. In some procedures fluid control 220 may be opened only long enough to relieve pressure in the fluid delivery system. This sequence causes the inflow to the delivery device to be vented through fluid control 220 and a more rapid pressure decrease on the delivery device. As described above the rapid pressure decrease helps minimize the amount of fluid leaked into the lumen, if desired. The dotted arrows indicate the directions of flows across the two valves. In some embodiments where relatively small amounts of leakage of the delivered agent into the body lumen is allowable, valve 220 may not be required.

An exemplary advantage in using a system shown in FIG. 2 is that because the high-pressure source holds therein multiple doses and the valve is operable at high rates, the system can be used for multiple fluid deliveries without re-filling.

In any of the embodiments herein, the fluid source maintained at a substantially constant high-pressure may be maintained at high-pressure by means of, for example without limitation, pneumatic, hydraulic, or mechanical means such as one or more springs.

FIG. 3 illustrates an exemplary high-pressure fluid source. The fluid source includes low-pressure fluid reservoir 340, high-pressure fluid pump 330, inline fluid control 310, and return valve 320. When idling, bypass fluid control 320 is open and inline fluid control 310 is closed. Fluid is then circulated through low-pressure 340 reservoir during idle. During an injection, fluid control 320 is first closed for a period of time generating high-pressure in the system to prime the fluid source. Fluid control 310 is then opened for an appropriate duration thereby delivering fluid at a rate consistent with the pump flow rate. Fluid control 320 is then opened and fluid control 310 is closed. In both of the described configurations the outflow resistance associated with the delivery device is much higher than the return path resistance. Pressure therefore drops rapidly in the outflow path when the bypass fluid control 320 is opened. This quick drop in pressure in the outflow path helps prevent leakage of the fluid agent into the lumen in which the medical device is positioned, if in fact this is desired.

Fluid controls as described herein can be any type of suitable valve, such as, for example without limitation, shuttle valves or poppit valves. In some embodiments the valves are actuated by interfacing a control interface with a system controller.

FIG. 4 shows an exemplary breadboard fluid control system configured for a pump source described in FIG. 3 that was used to investigate the characteristic associated with needle-less injections into renal artery tissues. The system is comprised of an outflow 401 for interfacing with a delivery catheter, pressure transducer 405 for monitoring the pressure at the outflow port 401, inline fluid control 410, bypass fluid control 420; low-pressure fluid reservoir 409, high-pressure pump source 408, controller interface 402, and a personal computer used as a controller (not shown).

FIG. 5 illustrates an exemplary embodiment of a high-velocity fluid delivery system adapted to deliver a fluid agent under high-pressure into a patient. System 500 includes system controller 510, delivery device 520, and delivery device control interface 530. The system controller may be a completely mechanical system or may comprise an electro-mechanical interface. The system controller (non-sterile) can be designed to be reusable, while the delivery catheter control interface and delivery catheter (sterile) can be designed to be discarded after a single use. In some embodiments, the features of the system controller, delivery device, and control interface are incorporated in a single disposable unit. Delivery device control interface 530 comprises an optional expandable member control interface, a fluid source, and a fluid control block. The expandable member can be in the form of a balloon, self-expanding structure, or any other suitable expandable or deformable member. In some embodiments the fluid source is a pump capable of delivering appropriate flows at the desired pressures as described herein, or a reservoir maintained at the appropriate operating pressure as described herein. Delivery device 520 is generally configured for endovascular or endoluminal delivery. Delivery device as used herein can be any type of suitable delivery catheter or other suitable medical device that can be positioned within a patient. The delivery device is shown including catheter shaft 521, the proximal end of which interfaces with delivery device control interface 530. The distal region of delivery device 520 comprises expandable member 523, radio opaque markers 524, a high-pressure delivery lumen (not shown), and features associated with facilitating rapid exchange on a guide wire. Delivery device also includes an aperture near expandable member 523 adapted to deliver fluid into the patient.

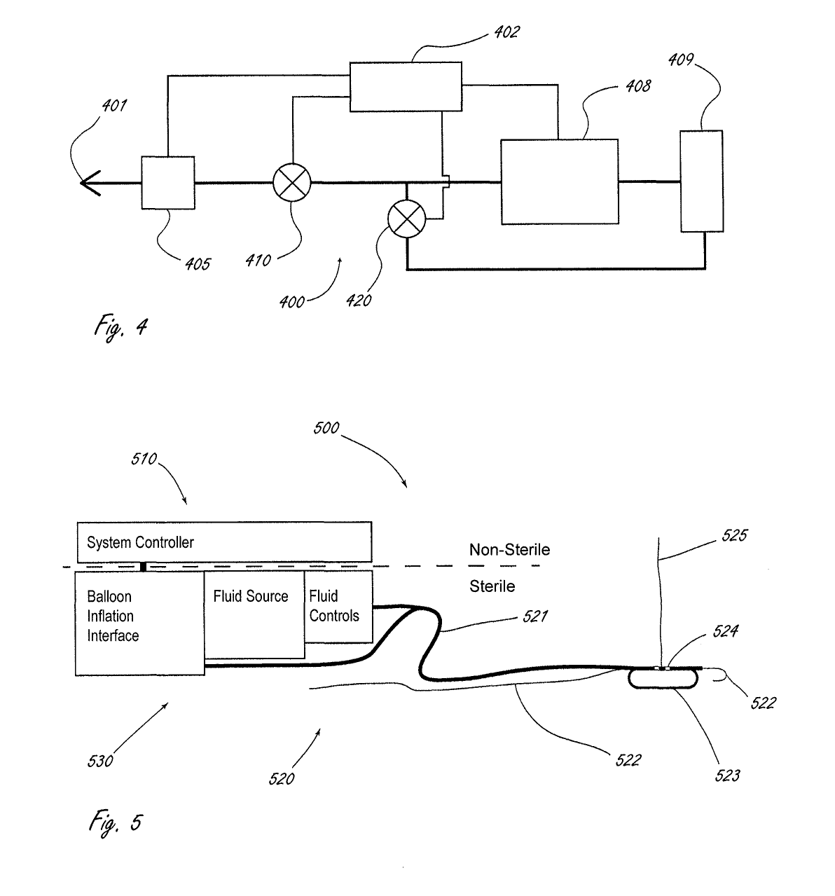

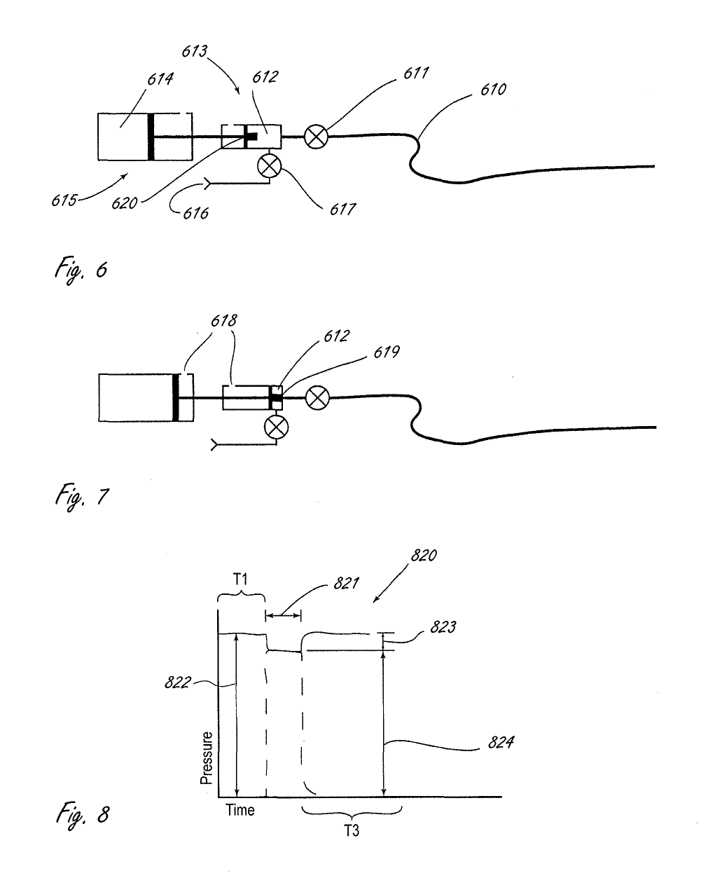

FIGS. 6 and 7 illustrate an exemplary high-pressure fluid source, which can be used as high-pressure fluid source 104 from FIG. 1. The high-pressure fluid source includes power source 615, fluid reservoir 613 with fluid 612 therein, outflow control valve 611, and delivery device 610. The fluid source also includes optional fluid input 616 and optional fluid fill valve 617, and vents 618 in both power source 615 and fluid reservoir 613 through which air is pushed or pulled depending on the use of the system. Power source 615 includes power mechanism 614, which in some embodiments can be a spring, compressed gas reservoir as shown, or other suitable mechanisms for generating power. Power mechanism 614 is adapted to push piston 620 distally within fluid reservoir 613 to maintain fluid 612 in fluid reservoir 613 under high-pressure while valve 611 is closed. FIG. 6 illustrates the system in a primed configuration, ready to delivery fluid 612. Fluid 612 is maintained under a pressure high enough to source an aperture in delivery device 610 at a pressure sufficient to allow for a high-pressure fluid agent injection. In use, after the system is primed as shown in FIG. 6, fluid control 611 is opened and fluid is delivered from reservoir 613, through open control 611, and through delivery device 610 and out an aperture in the delivery device (not labeled but described below). FIG. 7 illustrates the system at the conclusion of a high-pressure injection after the front face seal 619 of piston 620 has seated on the distal surface fluid reservoir 613 thereby cutting off the flow of fluid to delivery device 610. Fluid control 611 can then be closed in preparation for subsequent injections of fluid. In the embodiment in FIGS. 6 and 7 the reservoir houses fluid for one fluid delivery. The fluid delivery step involves delivering the entire volume of fluid housed in reservoir 612 at one time. The reservoir can subsequently be re-filled with fluid, either manually or automatically. The front face seal 619 in the embodiment in FIGS. 6 and 7 allows for precise control of delivered fluid volume in a system which only requires that valve 611 be opened rapidly. This is in contrast to the system of FIG. 2 in which valve 210 must be both opened and closed to facilitate a controlled volume of delivery. One exemplary advantage of the system in FIGS. 6 and 7 is primarily in the reduced complexity and therefore cost of the fluid control mechanisms.

FIG. 8 is a graph illustrating pressure vs. time and illustrates the pressure of the fluid within the fluid reservoir 613 in FIGS. 6 and 7, which is represented by the solid line, and the pressure of the fluid distal to fluid control 611, which is represented as the dashed line. Time epoch T1 is the time period after which the system has been primed (FIG. 6), and pressure 822 indicates the high fluid pressure of fluid 612 within fluid reservoir 613. Time epoch 821 indicates the period in which the high-pressure fluid is in communication with the delivery system 610, and pressure 824 is the high fluid pressure during the delivery phase. There is a negative pressure difference between time epoch 821 and time epoch T1. Time epoch T3 is the time period following the fluid delivery after seal 619 closes. During time epoch T3 the fluid pressure of fluid 612 within reservoir 613 returns to pressure 822.

The dashed line in FIG. 8 represents the fluid pressure at a location distal to fluid control 611. During time epoch T1, after the system is primed, this pressure is zero. During time epoch 821 when the fluid agent is delivered, control 611 is initially opened and fluid 612 is released under pressure from fluid reservoir 613. The fluid is forced down the fluid line lumen to the aperture. The pressure distal to fluid control 611 in time epoch 821 therefore increases abruptly to pressure 824, and after the fluid has been delivered from the aperture, as indicated in time epoch T3, the pressure distal to fluid control 611 drops abruptly back to ambient.

As can been in FIG. 8, there is a negative pressure change in the fluid in the fluid reservoir as the fluid delivery begins. This change can be made arbitrarily small by increasing the capacitance of power source 615. It is of note that a positive pressure transient is not created in fluid at the fluid source during the fluid delivery step because the fluid is primed to be under high-pressure. The velocity of the fluid delivered out of the aperture in the delivery device is sufficient to pierce tissue with minimal damage and yet expose the target tissue to a sufficient volume of tissue to disrupt the target tissue as needed.

As used herein, fluid that is “maintained” under high-pressure refers at least to the fact that the system is maintained in a primed state under high-pressure. When primed under high-pressure, a fluid control is then opened distal to the fluid reservoir to release the fluid primed and maintained under high-pressure. This is different than systems that generate a high-pressure transient at the fluid source and thereby do not require a control valve downstream the fluid reservoir.

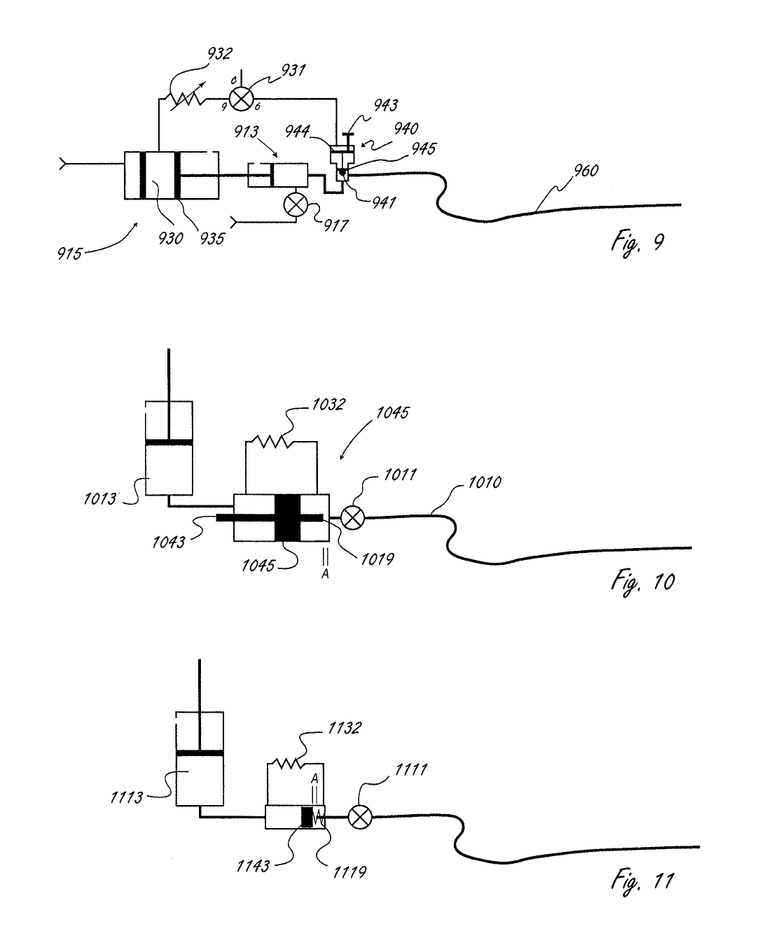

FIG. 9 illustrates an embodiment of a system in which an exemplary high-pressure fluid source 915 is coupled to elongate delivery device 960. In this embodiment the high-pressure source comprises a fluid reservoir adapted to house a volume of fluid sufficient for multiple discrete fluid injections and associated control mechanisms capable of controlling the volume of an individual injection. As shown primary power source 915 is pneumatically driven, but may be, for example, hydraulically or spring driven. Power source 915 comprises relatively low-pressure fluid source 930 that is used to power pilot valve 940. Pilot valve 940 comprises valve seat 941 adapted to interface with a high-pressure piston 945. High-pressure piston 945 is in turn coupled to low-pressure piston 944. The surface areas of pistons 944 and 945 are sized such that the pressure generated in the chamber at the valve seat 941 by pilot valve 940 is greater than the pressure generated in the high-pressure fluid source. Pilot valve volume adjustment is facilitated by volume adjustment 943. Low-pressure fluid in low-pressure fluid source 930 is communicated through adjustable fluid resistor 932 and 3-way valve 931 to the low-pressure side of adjustable pilot valve 940. Exemplary usage in the system is as follows. As the pressure generated by the low-pressure fluid source 930 on the pilot valve low-pressure piston 944 is sufficient to generate a pressure greater than that generated in the high-pressure fluid, the pilot valve is in the off, or closed, position.

FIG. 9 shows valve 940 in an open, or on, configuration. Before the fluid is delivered a delivery volume is defined by adjusting volume adjustment 943 some distance away from low-pressure piston 944 surface. When valve 931 is then momentarily reconfigured for flow from “b” to “a” to flow from “b” to “c”, the low-pressure fluid pressure drops to ambient on the low-pressure side of pilot valve 940. The pilot valve piston then shifts position until it encounters the volume adjustment 943 and the valve seat is opened. What is meant by momentarily in this context is a time sufficient for the pilot valve piston to shift to the fully open position. On re-attaining the default configuration of valve 931 where flow is “b” to “a,” low-pressure fluid begins to leak back into the low-pressure side of the pilot valve 940 at a rate defined by the value of the adjustable fluid resistor 932. The length of time to close the pilot valve 940 is therefore adjusted by both the length of travel (required volume) defined by adjustment of adjuster 943 and on the filling rate defined by fluid resistor 932. The delivered volume of fluid is therefore the volume associated with period during which the pilot valve is open. In alternative embodiments only one of the two controls 932 and 943 are included. In others one will be used as a calibration means and the other as a user control.

The embodiment in FIG. 9 can be modified to include a sensor such as a pressure transducer (such as the pressure transducer shown in the embodiment above in FIG. 4) or other means to infer velocity. The sensor can be added, for example, at valve seat 941. The sensor is adapted to provide feedback information indicative of the pressure differential across the delivery aperture, or the velocity of the fluid. An exemplary method of use compares the feedback data from the sensor with reference data to determine if the pressure is sufficiently high, or if the velocity is sufficiently high. If either parameter is not high enough damage may occur to the intermediate tissue, which can be disadvantageous when the intermediate tissue is, for example, an arterial wall. Alternatively, if either parameter is not high enough it can be determined that the fluid agent was not delivered at a high enough pressure or velocity and therefore did not adequately reach the target tissue (i.e., the target tissue was not adequately exposed to the fluid agent). If this is the case the method could include delivering one or more jets of fluid, and again determining if either the pressure or velocity were sufficiently high. In addition or alternatively to comparing the peak or plateau pressure to reference data, the time of the rise in pressure from baseline to peak or plateau can be determined and compared to reference data. When the pressure does not rise from baseline to peak or plateau quickly enough, damage to the intermediate tissue may not be minimized. In some embodiments it is determined if the rise in pressure occurs over a time longer than 15 msec, and in some embodiments over a time longer than 5 msec. If it does take longer than the reference time, feedback can be provided that indicates that, for example, the fluid delivery was ineffective or that damage occurred to the intermediate tissue. Towards this end it is also useful to purge the system with one or two test shots prior to deployment of the device adjacent to the target tissue. Doing so insures that air is not trapped in the system. Air trapped in the system can compress, and thereby slow the rise time of the pressure pulse.

FIGS. 10 and 11 illustrate alternative embodiments of alternate metering outflow valve variations. FIG. 10 illustrates valve 1045 secured to delivery device 1010. In FIG. 10 metering adjustment 1043 is linearly displaced an amount “A” such that linear displacement “A” equates to the expected delivered volume. Piston 1043 seals against the inner walls of valve 1045. Fluid resistor 1032 has very high fluid resistance and allows fluid to translate from one side of piston 1043 to the other as adjustments are made. A high-pressure source 1013 feeds fluid into metering valve 1045 on the upstream side of piston 1043. When control valve 1011 is opened a slight pressure differential develops across piston 1043 driving it to the right in the figure, closing fluid off at valve 1019. Fluid resistor 1032 is sized such that its resistance is sufficient to limit fluid flow from one side to the other at the change in pressure associated with the piston displacement during fluid delivery. In alternative embodiments the external resistor 1032 can be incorporated into piston 1043 or it can be inherent in the design of the interface between piston 1043 and the cylinder wall.

FIG. 11 illustrates an embodiment similar to the embodiment shown in FIG. 10. In the device shown in FIG. 11, when valve 1111 is opened, a small pressure differential is generated across piston 1143 by fluid resistor 1132. As in the embodiment of FIG. 10 the fluid resistor may be incorporated in the piston or the interface of the piston and the cylinder wall. When valve 1111 is opened, piston 1143 will travel distance A and seal against the distal end of the cylinder, thereby delivering a volume equivalent to distance A times the area of the cylinder. When valve 1111 is closed, pressure will equalize across piston 1143 and spring 1119 will return the piston 1143 to its primed position.

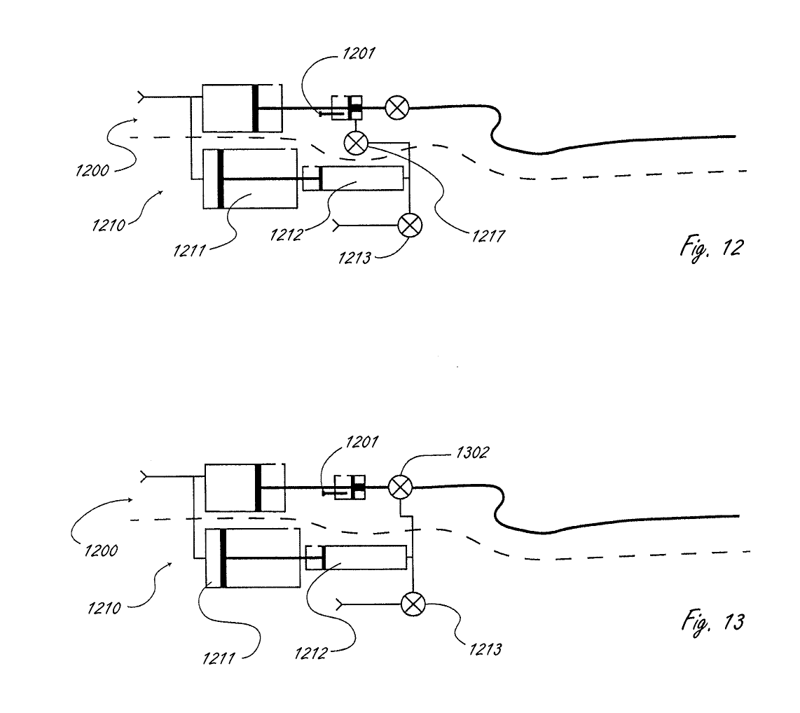

FIGS. 12 and 13 illustrate two variations of the system of FIGS. 6 and 7, which incorporate automatic high-pressure refilling systems. In FIG. 12, high-pressure delivery system 1200 is similar to the system of FIGS. 6 and 7 with the exception that volume control mechanism 1201 is incorporated in the high-pressure reservoir. High-pressure refilling system 1210 comprises a power source 1211 interfaced with a high-pressure fluid source 1212, which in turn is interfaced with high-pressure delivery system input valve 1217 and optional filling valve 1213. High-pressure refilling system 1210 is configured such that the pressure within high-pressure refilling reservoir 1212 is maintained at a pressure somewhat greater than the pressure in the high-pressure delivery system 1200. In use, volume adjustment mechanism 1201 is adjusted to the appropriate volume. Valve 1217 is then opened allowing fluid to pass from the refill reservoir to the high-pressure delivery reservoir. Valve 1217 is then closed and the high-pressure delivery system is ready to use. Optional valve 1213 may be used to fill the refilling reservoir. As depicted in FIG. 12 the power source 1211 is a low-pressure pneumatic drive where the drive pressure will be equivalent to the low-pressure drive pressure times the ratio of the surface areas of the power source piston/high-pressure refilling reservoir. In FIG. 13 the high-pressure delivery system input valve 1217 has been replaced by a three-way valve 1302, but other similar components are similarly labeled.

The delivery devices described herein, which are indirectly or directly coupled to the substantially constant high-pressure fluid source, have at least one aperture therein adapted to allow a fluid agent to be delivered from the fluid source and out of the aperture under high-velocity.

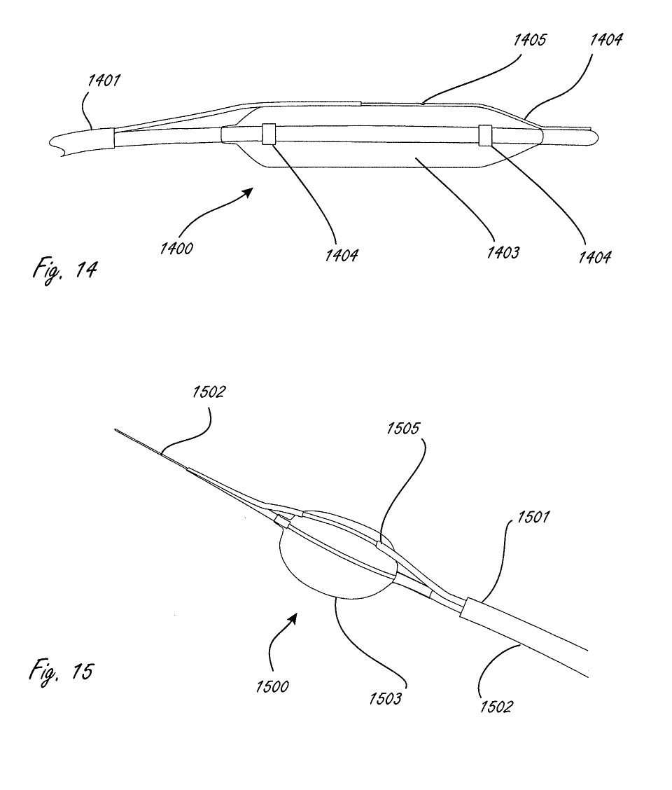

FIGS. 14 and 15 illustrate two exemplary distal regions of two exemplary delivery devices. FIG. 14 illustrates a distal region of a deliver device 1400 that includes an over-the-wire configuration for delivery. The delivery device includes catheter shaft 1401, comprising high-pressure fluid delivery line 1405, expandable member 1403, a guide wire lumen (not labeled), balloon inflation lumen (not labeled), and radio opaque markers 1404. Expandable member 1403 is shown as a rigid 20 mm long and 6 mm diameter cylindrical balloon but can have other configurations, and is secured to the outer surface of the distal region of catheter shaft 1401. High-pressure fluid line 1405 has at least one aperture formed therein in its distal region, and is secured to expandable member 1403 such that a fluid jet aperture (which is not visible but is included in the device) faces (i.e., opens) radially outward from the long axis of the expandable member 1403. The aperture can be anywhere along the length of fluid line 1405, but in this embodiment is positioned at the longitudinal center of expandable member 1403.

In an exemplary use, the delivery device is primed with fluid so that fluid is disposed in the delivery device fluid delivery line. A delivery catheter, examples of which are well known, is advanced to a region of interest within the patient. A guidewire is then fed through the delivery catheter to the distal end of the delivery catheter. Alternatively, and more commonly the guide wire is delivered to a location adjacent to the target tissue, then the delivery catheter is advanced over the guidewire near the target location. Delivery device 1400 is then advanced over the guidewire with the guidewire disposed in the guidewire lumen. Once in the desired position, delivery device 1400 is moved distally relative to the delivery catheter. Catheter shaft 1402 is advanced to position the jet aperture adjacent to the target tissue (and directly adjacent and engaging the intermediate tissue). Expandable member 1403 is inflated with fluid advanced through the inflation lumen in catheter shaft 1402. A high-velocity jet of fluid agent is then delivered as described herein.

Three radio opaque markers 1404 are also incorporated into the distal region of the delivery device. The two markers 1404 on catheter 1402 delineate the axial location of the fluid jet aperture, and the most distal marker 1404 provides information on the radial orientation of the aperture.

In some embodiment the high-pressure delivery line, or lumen, is substantially flush with the outer surface of the balloon (or other expandable member). In these configurations the high-pressure lumen does not extend further radially than the outer surface of the balloon. This configuration provides better engagement between the balloon and the lumen wall in which the balloon is disposed and expanded. This provides a better seal between the balloon and the lumen wall, which reduces the likelihood of fluid leaking back into the lumen once it is delivered out of the aperture. In some embodiments the high-pressure delivery lumen is integrated into the balloon structure. This can be accomplished by incorporating one or more lumens into the extrusion used to form the balloon. The lumens are maintained during the balloon forming process and the resulting balloon structure would therefore include one or more integrated high-pressure delivery lumens. In some embodiments a channel is formed in the balloon to accommodate the high-pressure fluid lumen. For example, a channel with a general “U” cross sectional shape is formed in the balloon, and the high-pressure lumen is secured within this channel. The high-pressure lumen is therefore substantially flush with the outer surface of the balloon.

FIG. 15 shows an alternate embodiment of a distal region of a delivery device similar to that shown in FIG. 14 and comprising the features of a rapid exchange guide wire configuration. Guide wire 1502 is shown entering the catheter shaft on the proximal side of balloon 1503 and exiting the shaft on the distal end of delivery catheter 1500. The expandable member 1503 in this embodiment is a generally spherical inflatable elastomeric balloon. High-pressure delivery line 1505 is secured to the surface of the balloon as described above in the embodiment in FIG. 14.

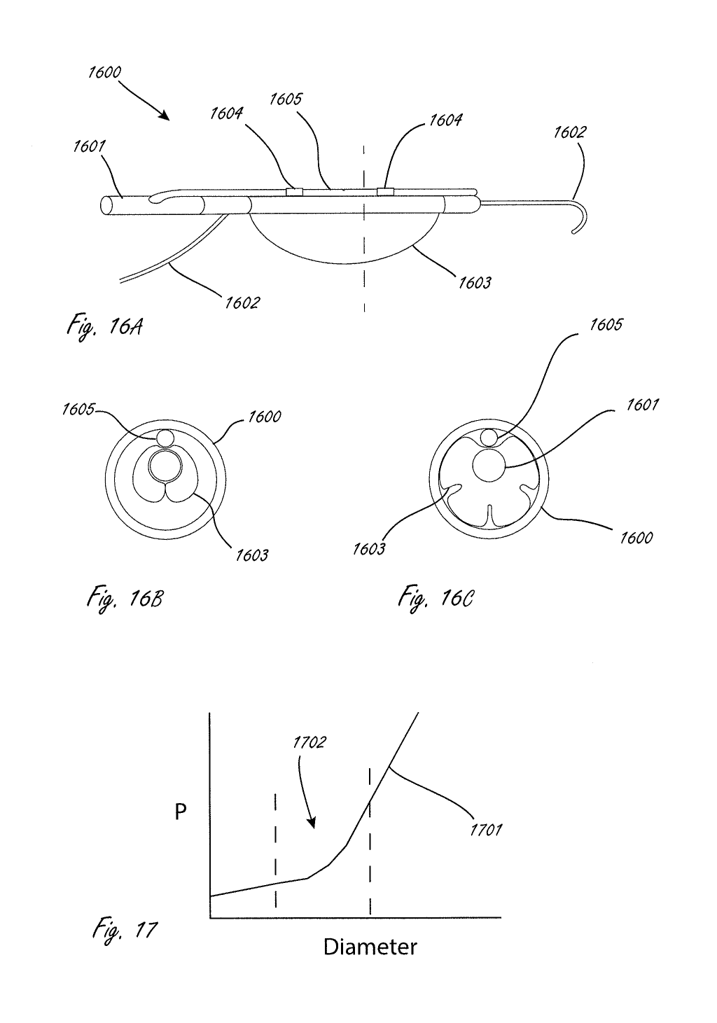

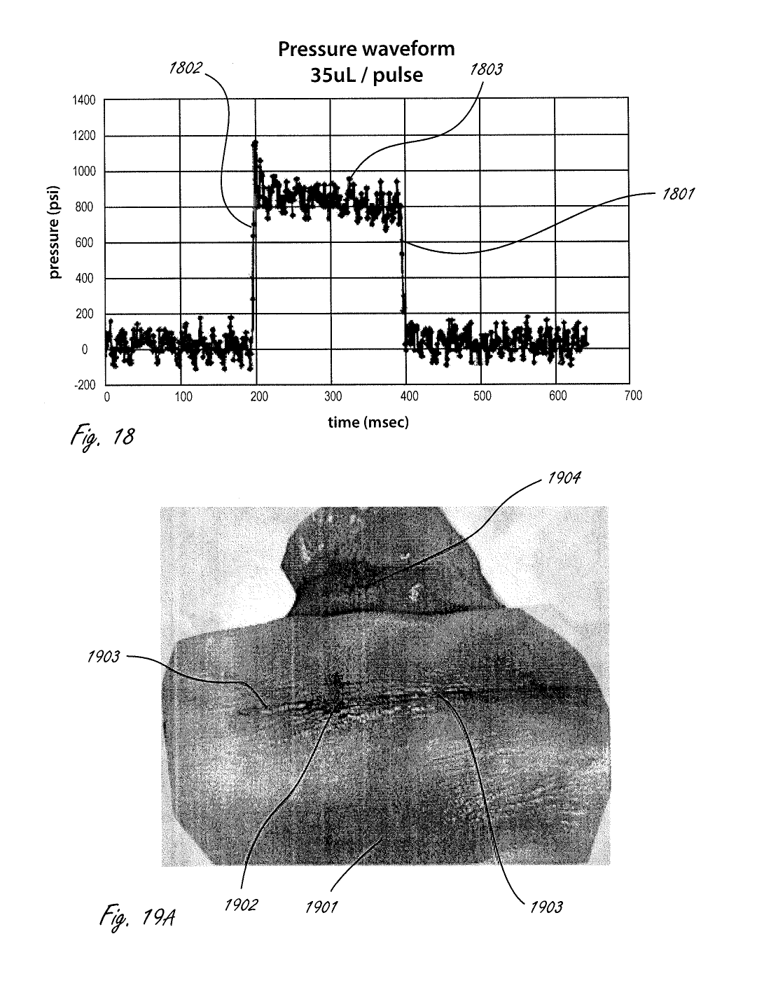

In an alternative design similar to those shown in FIGS. 14 and 15, the balloon is radially offset relative to the expandable member shaft such that the high-pressure line has a substantially straight configuration across the surface of the balloon when the balloon is expanded. The embodiment in FIGS. 16A-16C enhances the precision with which interface pressure can be measured and controlled. The embodiment in FIGS. 16A- 16 C includes balloon 1603 that is radially offset with respect to catheter shaft 1601. High-pressure fluid delivery line 1605 is secured to balloon 1603. High-pressure line 1605 also includes radio opaque markers 1604. The embodiment comprises a rapid exchange guide wire interface demonstrated by the path of guide wire 1602. Balloon 1603 is carried on catheter shaft 1601 which may incorporate a braid or other stiffening elements to facilitate larger torque carrying capacity. General features of the catheter shaft are not shown. FIG. 16B illustrates a cross section of the delivery device of FIG. 16A configured for delivery and prior to inflation, wherein the delivery device is positioned within vessel 1600. In this configuration balloon 1603 is deflated and folded. FIG. 16C represents the balloon in its inflated state where the balloon has a larger diameter then the vessel 1600 in which it is expanded. In such a configuration the pressure required to expand the balloon will be minimal, and the pressure monitored during inflation will be indicative of that associated with stretching the vessel wall. By recording volume versus pressure the diameter pressure curve of FIG. 17 can be calculated and a desired pressure range can be determined. Such a system can be used to identify the appropriate inflation pressure by monitoring the relative change in modulus as opposed to targeting a particular absolute pressure.

The systems and devices are adapted to be used to deliver a fluid agent to target tissue that is more distant to the aperture than tissue directly adjacent the aperture. The systems can be used to minimize the damage done to the intermediate tissue, and one manner in which this can be accomplished is with fluid delivered at high-velocity out of the aperture. An exemplary use is to position the delivery device within a renal artery and deliver a fluid agent out of an aperture at high-velocity. The fluid passes through the wall (with minimal damage to the intermediate wall tissue) to a location where it can interact with neural tissue surrounding the renal artery. The interaction of the fluid and nerves disrupts the neural transmission along the nerves, reducing hypertension. Methods of reducing hypertension with a fluid agent delivered out of a delivery device under high-velocity are described in U.S. Pat. App. Pub. No. 2011/0257622, filed Mar. 24, 2011, the disclosure of which is incorporated herein by reference. As described above and shown in U.S. Pat. App. Pub. No. 2011/0257622, the fluid agent is delivered out of the delivery device, pierces through the renal artery lumen wall, and is exposed to target neural tissue more distant from the lumen to disrupt neural transmission along the nerves and reduce hypertension. The systems, devices, and methods herein provide sufficient penetration of the fluid through the renal artery such that neural tissue is exposed to the fluid, while minimizing the amount of fluid that is leaked back into the renal artery, and thus the vasculature. The systems, devices, and methods herein also provide fluid penetration through the renal artery such that the injury associated with the fluid penetration is minimized at the luminal entry point.

In some systems previously described in the patent literature, the fluid pressure within the fluid source is relatively low prior to and after fluid delivery into the patient, but may be relatively high during fluid delivery and immediately prior in time to the delivery of the fluid. An exemplary disadvantage to these systems is that if the fluid pressure is initially too low, the fluid may not be delivered far enough into the target tissue. For example, in systems use to deliver fluid from the renal artery and into neural tissue surrounding the renal artery to disrupt neural transmission along those nerves, the fluid may ultimately be delivered only partially into the medial layer, when the desired outcome is that the fluid is delivered completely through the medial layer, in which the target nerve tissue is disposed. An additional exemplary disadvantage to these systems is that, because the pressure will drop back down to the relatively low-pressure, if the pressure drops off too quickly, the fluid might not penetrate all the way through the medial layer, which is undesirable for reasons set forth above. By maintaining the fluid pressure within the fluid source at a substantially high-pressure, the fluid pressure doesn't return to a relatively low-pressure, but rather is maintained at the substantially constant high-pressure. The potential problems of not penetrating deep enough into the medial layer, and thus failing to sufficiently disrupt neural transmission along the neural pathway, are therefore eliminated.

By delivering a pressure pulse and thereby a fluid stream with rapid rising and falling mean velocity, the fluid, when delivered, will both penetrate through the lumen to surrounding tissue with minimal injury to the tissues at the entry point and minimize leakage of the fluid back into the lumen.

FIG. 18 illustrates the pressure waveform generated in the system from FIG. 4 when using a jet aperture of 1.5 mil diameter, as measured in the pressure transducer 405. The delivery volume was approximately 35 uL delivered over a period of approximately 200 msec. The pressure transient, as measured at pressure transducer 405, associated with the increasing pressure 1801 occurred over a period of approximately 5 msec, and the pressure transient associated with the release of pressure 1802 occurred over a similar time frame. The pressure pulse attains a relatively constant plateau pressure of approximately 900 psi.

In some embodiments the diameter of the one or more fluid jet apertures is between about 1 and about 5 mils. In some embodiments the velocity of the fluid jetting from the medical device is between about 50 and about 400 m/sec. In some embodiments the flow rate of the fluid from the constant high-pressure source is between about 5 and about 40 mL/min. In some embodiments the duration of the fluid pulse is between about 50 and 500 msec. In yet other embodiments the duration is multiple seconds. In some embodiments the volume of fluid delivered per pulse is between about 10 uL and about 500 uL. In yet other embodiments the delivered volume may be multiple mL's. In some embodiments the time of the transition between the baseline pressure and the elevated pressure, and the time of the transition between the elevated pressure and the baseline pressure (e.g., transitions 1801 and 1802 in FIG. 18) is less than about 15 msec, and may be less than 5 msec, and additionally may be less than 1 msec. In general, shorter transition times translate into more efficient penetration and less fluid leaking into the lumen.

As used herein, high-pressure refers to pressure above about 750 psi, and includes pressures between 750 psi and 5000 psi. The systems are adapted to maintain the fluid in the fluid reservoir in the high-pressure fluid source under pressures of about 750 psi and about 5000 psi.

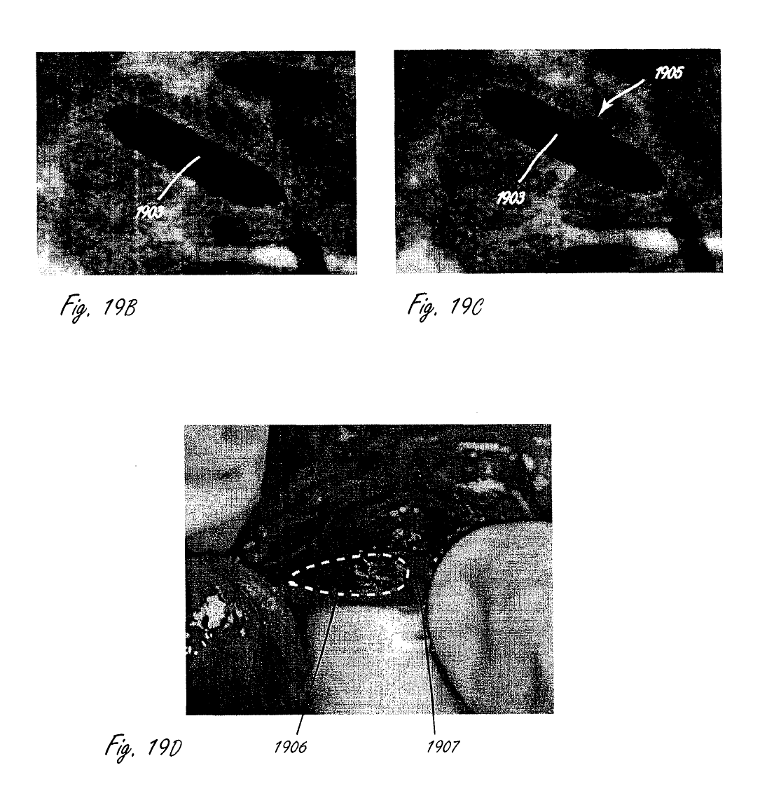

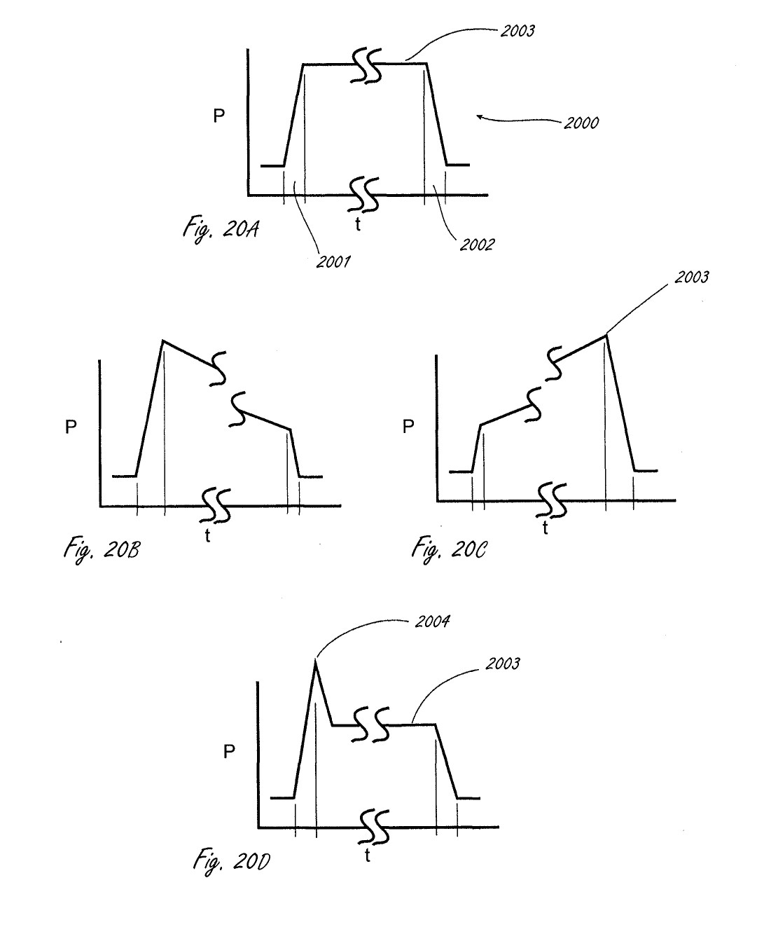

FIGS. 19A-19D show various images of tissue treated with fluid injections exhibiting a pressure pulse similar to that illustrated in FIG. 18, delivered with the system shown in FIG. 4 and the delivery catheter shown in FIG. 14. FIG. 19A shows the luminal surface 1901 of a sample of porcine renal artery tested in vitro that has been split after the injection such that the entry injury can be viewed. The injectate comprised a blue dye. The injection site is indicated by 1902 and distinguished by the darkening from the dye. The visibly stained area on the luminal surface is approximately 2 mm long in the radial direction (vertical in image) and about 0.5 mm wide. Darkened area 1903 corresponds to the location of the high-pressure delivery line 505. Periventricular adipose tissue darkly stained with injectate is visible at 1904. FIGS. 19B and 19C show fluoroscopic images taken during an in vivo porcine study. Balloon 1903 is visible via contrast agent which has been used to inflate the balloon. The balloon is shown in the renal artery where it has been delivered via an endovascular approach. In this study the injectate contained both a fluoroscopic contrast agent and a blue dye. FIG. 19B shows the balloon and surrounding tissue just prior to an injection. FIG. 19C shows the balloon and surrounding tissue just after an injection. The injectate is visible in FIG. 19C at 1905. FIG. 19D is a photograph from the necropsy of the same treatment zone from another animal. Darkened area 1906 within the dotted line shows the stained injury zone in contrast and beside a non-injured zone 1907 on a renal artery.

FIGS. 21A and 21B are fluoroscopic images and illustrate the cloud of a 70% ETOH/30% Contrast injectate, where the delivery parameters were 1.5 mL over 9 seconds at approximately 80 m/sec, facilitated by a 1200 psi pressure pulse through a delivery system similar in configuration to that of FIG. 15. A dashed white line has been drawn to highlight the injectate cloud 2110. A guide wire 2101 can be seen extending through a renal artery of a pig and delivery catheter 2100 can be seen at the bottom right in the figures. Radio opaque marker 2102 located adjacent the injection aperture is visible within the contrast cloud. FIG. 21B is a view of the same injectate cloud from a different angle which demonstrates a greater than 180 degree radial spread of injectate around the long axis of the renal artery. Inflatable balloon 2103 is visible in FIG. 21B.

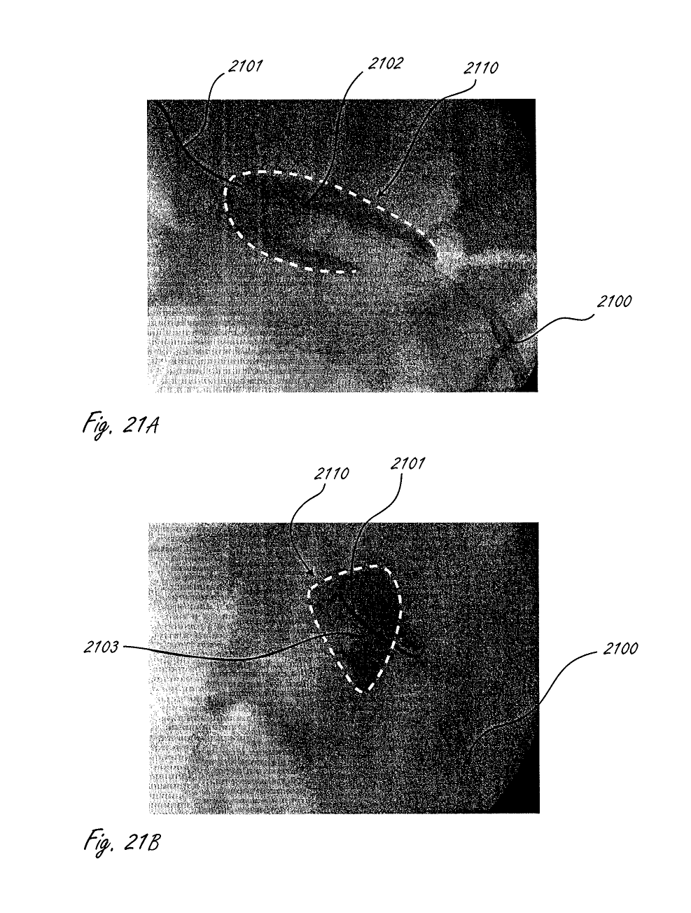

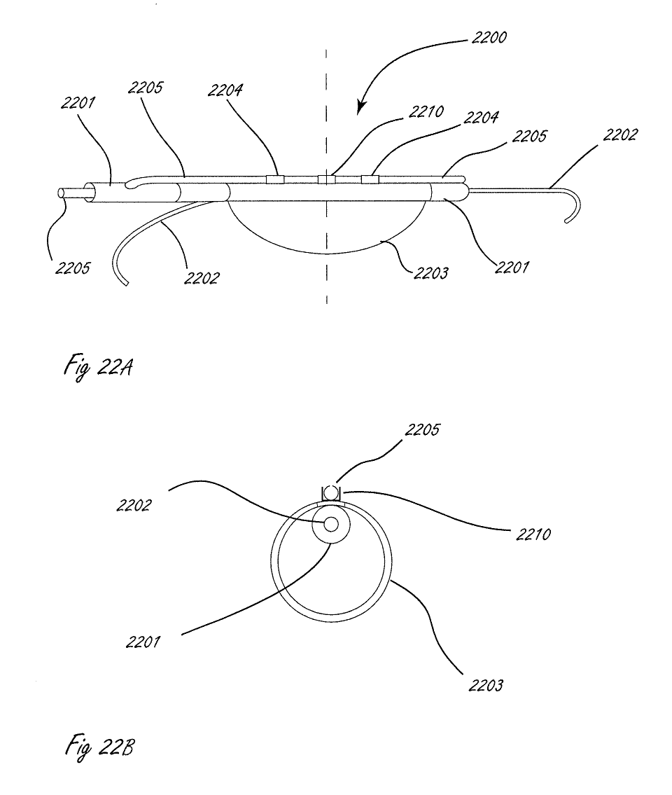

FIGS. 20A-20D illustrate different generalized waveforms 2000 useful in needle-less injection of fluids into periluminal spaces. FIG. 20A represents the type of waveform depicted in FIG. 18 where the region between the rising and falling transitions 2003 is relatively flat. Exemplary features include the rapid transitions associated with the onset of the pressure pulse and the decay of the pressure pulse. A rapid onset pressure transition 2001 is important in creating a well-defined injury of minimal size wherein the injectate is primarily delivered through the injury with very little leakage around the injury entry surface. Similarly a very rapid final decay transition 2002 is important in minimizing leakage of fluid around the injury entry surface. When it is required that the low-pressure leakage be minimized on the pressure decay portion of the pulse it is useful to create the jet aperture adjacent to the distal plug in the high-pressure delivery line. In this fashion entrapped air will be washed out easily during priming prior to actual jetting. If this step is not performed, air may be trapped distal to the jet orifice, and compressed during the pressure rise portion of the jet cycle. On pressure decay this air will re-expand and force a small volume of injectate out through the jet orifice. This is of primary importance when the injectate is comprised of very toxic or ablative materials and minimizing injury to non-target tissues is required. Transition times should be at least less than 15 msec and preferably less than 5 msec as demonstrated in the experiments described herein, and optimally less than 1 msec. Apart from leakage, a sharp rising edge facilitates better penetration. Once an entry injury has been created it is often the case that pressure can be dropped and injectate will spread on the distal side of a well-defined puncture injury. In such a procedure, injury to the tissues at the entry site associated with the injectate can be minimized while larger volumes of injectate can be delivered deeper into the tissue without increasing the depth of injury. FIGS. 20B and 20C illustrate two pressure waveforms useful in producing such injuries. In FIG. 20B, after the peak pressure is attained the pressure is allowed to trail off via a ramp to a pressure still sufficient to penetrate through the entry injury. At the end of the pulse the pressure is rapidly dropped for the reasons set forth here. FIG. 20D is similar to that of FIG. 20B except that as opposed to ramping down pressure an initial short high-pressure peak 2004 is used to create the injury, which is then followed by a lower pressure plateau of sufficient pressure and duration to deliver the requisite volume of injectate to an appropriate depth via the entry injury. In some situations it may be useful to spread that injectate more evenly through the depth of tissue, in which the pulse of FIG. 20C could be desirable. Alternatively the volume of injectate may be additionally regulated by delivering multiple pulses at a specific location, wherein the pulses may be comprised of various combinations of those described herein and/or various delivery velocities.

With reference to the treatment of hypertension by renal nerve ablation (examples of which are described in more detail in U.S. Pat. App. Pub. No. 2011/0257622), the volume of injectate delivered may be increased via multiple injections in a single location or multiple injections in multiple sites, or a large volume delivered to one site and allowed to spread. When delivering injectate at one site via multiple injections, the spreading of the injectate may be monitored by fluoroscopy when a contrast agent is comprised in the injectate. The number of injections may be controlled by watching how the injectate spreads under fluoroscopy, and stopping the procedure when the desired spread has occurred. When injecting at multiple sites a device such as that of FIG. 15 may be relocated for each injection or alternatively a device similar to that of FIG. 14 may incorporate multiple parallel injection systems, wherein each line is coupled to a single fluid source or individual fluid sources. Devices described in U.S. Pat. App. Pub. No. 2011/0257622 can also be modified to be used with any of the system components described herein and according to any of the methods herein.

FIG. 17 illustrates a typical pressure diameter profile associated with an artery. An appropriate pressure at the interface between the jet aperture of the medical device and the luminal wall is important when minimal injury at the luminal surface of the vessel and control of the depth of injectate delivery is desired. The greater the interface pressure, the smaller the luminal injury and the greater control of penetration depth. However, if the interface pressure is increased too much the vessel may be injured. A balance must therefore be reached between interface pressure vessel distension. A typical vessel exhibits a low modulus during initial extension, begins to stiffen, and then exhibits a much higher modulus. As the vessel is extended further into the high- modulus region the tissue will be damaged. Region 1702 indicates a target region of interface pressure where damage to the vessel can be minimized and interface pressure is high enough to create a clean puncture of the lumen wall.

In the embodiments illustrated in FIGS. 14 and 15, high-pressure delivery lines 1405 and 1505 have a 14 mil outer diameter and 12 mil inner diameter polyimide tube. The delivery apertures, not visible in the figures as they are too small, are 1.5 mil. The total length of the delivery lines is approximately 32 inches.

The following describes the expected fluid dynamic behavior for a fluid delivery system that includes a long fluid pipe with an exit aperture near the distal end, as do the embodiments in FIGS. 5 and 6. The description particularly applies where the fluid delivery line has an inner diameter of approximately 12 mil and the delivery aperture is in the range of about 0.5 to about 5 mil, or more particularly about 2 mil. For such systems the fluid velocity will be described by the equation:

v(P,Beta,ρ)=C_{d}*(1/(1-Betâ4))̂0.5*((2*P)/ρ)

Where P is the pressure differential across the exit aperture, Beta is the ratio of the diameter of the delivery tube inner diameter/diameter of the aperture, ρ is the density of the delivered fluid and C_{d} is the coefficient of discharge. Experimental data collected demonstrates a value for C_{d} in the range of about 0.5 to about 0.8 with a value of about 0.65 being typical for the configuration listed above. Experimental data collected from such a system demonstrated 1.5 mL delivered in 9 seconds through a 2 mil diameter exit aperture at 1200 psi, using a delivery fluid with a density of approximately 1.1 gm/mL. Using the relation average_velocity=Volume_delivered/(duration*Area_aperture), this implies an average delivery velocity of 82 m/sec. Using the functional relation described above and a C_{d} of 0.65, the average fluid velocity would be approximately 78 m/sec at 1200 psi as measured at the exit valve. Given the expected pressure loss across the 32 in long, 12 mil diameter delivery tube at the average flow rate, this would imply a pressure differential of approximately 1135 psi across the exit aperture.CO₂ cartridges provide a means for maintaining a constant pressure within the constant pressure source as the internal pressure in a CO₂ cartridge will remain relatively constant at a given temperature as long as there remains a mixture of gas and liquid within the cartridge. Pressure could hence be adjusted by adjusting the temperature of the cartridge. The following table lists the internal pressure as a function of temperature for a CO₂ cylinder containing CO₂ in both liquid and vapor phases.

TABLE 1

| Temperature (F.) | Pressure (psi) |
|---|---|
| 80 | 969 |
| 70 | 853 |
| 60 | 747 |
| 50 | 652 |

Exemplary fluid agents that can be delivered, such as to treat neural tissue peripheral to body lumens, using any of the methods, systems, and devices herein, can be found in U.S. Pat. App. Pub. No. 2011/0257622, U.S. Pat. App. Pub. No. 2011/0104061, and U.S. Pat. App. Pub. No. 2011/0104060, the complete disclosures of which are incorporated by reference herein.

In some embodiments the systems herein can be used to ablate target tissue. When performing localized ablations of tissue, it is often advantageous to use an ablatant that is chosen to specifically target a particular tissue or tissue function, and to impart minimal effects on adjacent tissues. In all cases the residence time of an ablatant cocktail will be dependent on the rate of its removal by normal body functions which include uptake by the capillary bed and the lymphatic system. When using a well targeted ablatant it will often be the case that it will have very little effect on the tissues associated with the normal removal processes. In such cases, the body will remove the ablatant as efficiently and quickly as possible. In such a situation it will be of great advantage to add to the ablatant cocktail some non specific ablatant, or an ablatant specifically targeted to impede capillary and or the lymphatic uptake to slow the body's ability to remove therapy targeted ablatant and thereby increase its residence time and thereby the magnitude of its effect for a given delivered volume and concentration.

Use of ablatants targeted at neural function such as guanethidine, reserpine, tetrodotoxins, botulinum toxin, or other ablatants have particular significance in the treatment of hypertension, such as in the ablation of renal nerves. These ablatants may have some effect on capillary uptake but should have little to no effect on lymphatic uptake.

It has been recently noted under by fluoroscopy that there is a significant increase in residence time for a contrast agent that has been injected in combination with a general ablatant such as ethanol (ETOH) vs. the same contrast agent which was injected in combination with saline. In these experiments a cocktail comprising 30% Ultravist 300 (a contrast agent) and either 70% ETOH or 70% saline by volume were observed over time for decay in contrast as measure fluoroscopically. The observation was that the contrast was observable for a longer period of time in the surrounding tissues when injected with ETOH as compared to when injected with saline. The general ablatant increased the residence time for the contrast agent compared to saline.

One aspect of the disclosure is a method of treating hypertension (e.g., but not limited to, from within the renal artery, such as in the applications incorporated by reference herein) by delivering a cocktail of a general ablatant (e.g., ethanol, glacial acetic acid, etc.) and an ablatant targeted at neural function. The targeted ablatant can be any of those listed herein. In one embodiment the cocktail comprises ethanol as the general ablatant and guanethidine as the targeted ablatant. The general ablatant will increase the residence time of the guanethidine and achieve a more successful ablation of the renal nerves.

One aspect of the disclosure is a method of treating hypertension by sequentially delivering a relatively smaller amount of a general ablatant, followed or preceded by delivery of the targeted ablatant. The general and targeted ablatants can be any of those described herein or any other suitable ablatants. The amount of general ablatant will be an amount smaller than is typically delivered to ablate the nerves, but is sufficient to increase the residence time of the targeted ablatant by inhibiting the body's ability to clear the targeted ablatant.

One aspect of the disclosure is a method of treating hypertension by delivering a cocktail of an ablatant targeted to neural function and an ablatant specifically targeted to impede capillary and/or the lymphatic uptake to slow the body's ability to remove therapy targeted ablatant. In this aspect a general ablatant could also be added to the cocktail in even smaller amounts than in the previous aspect.

FIG. 22A illustrates the distal end of a high-pressure injection delivery device comprising comparable features and function to that described in FIG. 16A. These include catheter shaft 2201 carrying high-pressure delivery line 2205, expandable member 2203, and radio-opaque markers 2204. The high-pressure delivery line 2205 comprises a fluid delivery aperture at the level of the dashed line for delivering fluids at high-velocity to a target tissue surrounded by non-target tissues. The expandable member comprised of a balloon in this embodiment is used to hold the high-pressure injection aperture against tissue adjacent to the target tissue. Also comprised in the high-pressure injection delivery device is an over the wire guidewire channel with guidewire 2202 shown within. The device of 22A additionally comprises an ultrasonic transducer 2210 which is used to characterize the thicknesses of the adjacent tissues and locations of tissue boundaries. In addition, the ultrasonic transducer can be used to monitor the location and boundaries of an injectate with in a tissue. In some embodiments this can be done during an injection, while in others it can be done during or after completion of an injection. FIG. 22B illustrates a cross section of the delivery device at the level of the dashed line.

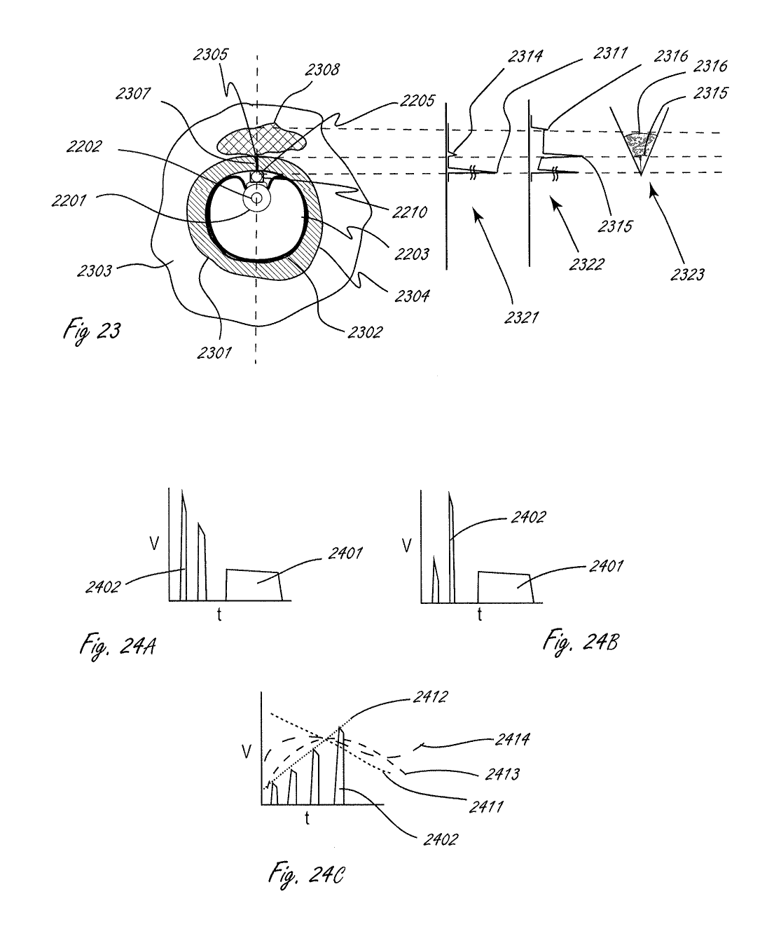

FIG. 23 illustrates the cross section of the device 2200 within a tissue volume during or after a high-pressure injection. The tissue illustrated is that of a typical artery comprised of epithelial layer 2301, a smooth muscle layer 2302, and a surrounding adventitial layer 2203. Additionally labeled are tissue boundaries 2304 between smooth muscle tissue and adventitial tissue and 2305 between tissue comprising injectate 2308 and adjacent tissues. The channel bored by the high-pressure injectate is represented by 2307.

Adjacent to the physical representation in FIG. 23 are graphics 2321 and 2322 representing what the a single element transducer 2210 would see at various stages of an injection. Graphic 2321 illustrates the transducer output signal prior to an injection where the boundaries between the epithelial layer and the transducer, in this case equivalent to the position of the epithelium, is seen as the peak 2311 followed by the peak 2315 corresponding to the boundary 2304. Graphic 2322 illustrates the transducer output signal during or after an injection. Here peak 2315 associated with boundary 2305 is seen at roughly the same location as that of 2314 in the pre injection image since the injectate boundary is at the same location. Following peak 2315 is the peak 2316 corresponding to furthest injectate boundary relative to the transducers field of view.

Graphic 2323 illustrates a 2D image of at a post injection time associated with a transducer 2210 comprising multiple elements used as a phased array.

FIGS. 24A-24C illustrate a number of alternate velocity time waveforms comprised of pulses, as opposed to continuous pressure time waveforms as illustrated previously in FIGS. 20, usable as control outputs in the present invention. Peak fluid velocity will be function of pressure, where velocity will in general increase with increasing pressure. Such outputs are comprised of low volume long pulses 2402 and long duration pulses 2401. In general, short pulses can be used initially to bore a tract to an appropriate depth and then longer duration pulses used to deliver an appropriate volume once an appropriate depth has been defined.

Outflow velocity of the injectate increases with pressure. A series of short pulses can be delivered at different pressures thereby effecting the outflow velocity of an injection. The depth of penetration can thereby be adjusted until an appropriate depth has been identified using feedback from the transducer. Upon identifying an appropriate depth, a lower-pressure higher-volume pulse can be delivered to complete the delivery of an appropriate volume as illustrated in FIG. 24B. In some instances, an initial pulse will bore too deeply and will require a follow-on lower-velocity pulse to attain the proper depth. In some instances, the injection port can be moved a small distance prior to delivery of the second pulse. This procedure can by repeated until an appropriate depth is defined and then followed with a long lower pressure peak 2401.

FIG. 24C illustrates a number of possible variations for how the peak velocity per pulse may be changed over time. The dashed lines indicate the variation in peak over time where 2412 shows a set of pulses commensurate with a curve 2412, the series of pulses comprising a set of monotonically increasing pulses 2402. Pressure pulses associated with the other variations have not been illustrated.

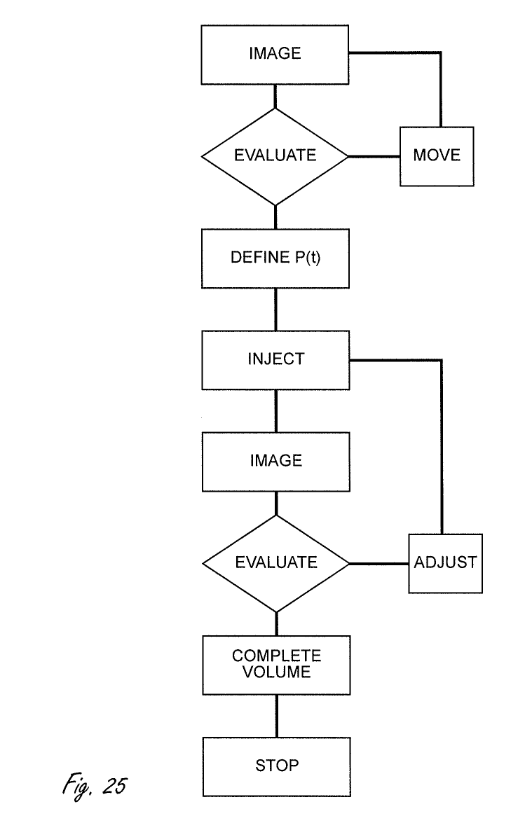

FIG. 25 illustrates one possible procedure for targeting injectate to a target tissue after an initial placement of the high-pressure injection delivery device, as follows. Adjacent tissues are imaged. The image is then evaluated. Evaluation can comprise evaluating tissue types, boundaries, boundary depths, and location of a target tissue and its depth. An assessment can be made as to whether there are any tissues between the target tissue and the injection site which would render the injection site less desirable. Such tissues could be dense tissues high in collagen such as cartilage, or high in calcium such as plaques, or any of many other possibilities known in the art. If such a tissue is identified the delivery device may be moved to a site where such tissue does not intervene between the target tissue and the injection site. Once a proper site has been identified any combination of the types, thicknesses, and depths of tissue and tissue boundaries are used to estimate a pressure time waveform to control the injection. An injection is then started. Once a portion of the injectate has been delivered, a new image is acquired and the image is evaluated for the location of the injectate. The volume of the injectate delivered is recorded. Given the location of the injectate the P(t) delivery waveform can be modified if required or desired. In some embodiments this process may be done under control of the user, while in other embodiments it may be automatic and done under control of a controller, such as by an algorithm stored on a storage medium. An additional portion of the injectate can then be delivered. If no adjustment for targeting the target tissue is required, the remaining volume can be delivered.

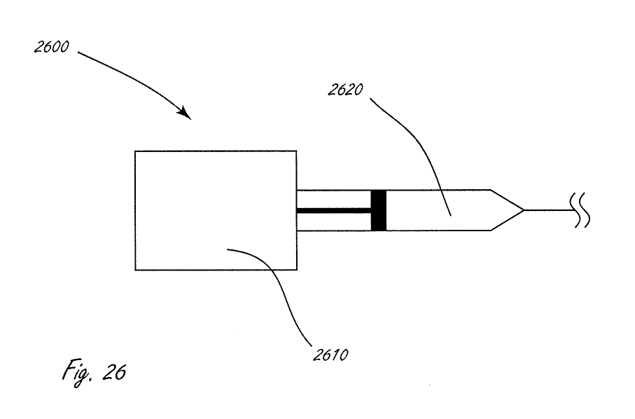

FIG. 26 illustrates a conventional pressure pulse sourcing system 2600 comprised of a syringe 2620 affixed to a controller 2610 for driving the plunger at high speed incremental amounts. This is in contrast to previous controllers described herein which meter out, via a control valve, fluids which are maintained at high-pressure within a high-pressure reservoir.

In yet another embodiment a transducer may be placed around the high-pressure delivery tube or with in the syringe in FIG. 26 (not shown) and the sounds associated with the delivery of fluid may be used as a control input in a high-velocity fluid delivery control loop.

1. A method of intravascularly imaging a high-velocity intravascular fluid injection, comprising:
positioning a fluid delivery catheter within a lumen within a patient's body, the fluid delivery catheter comprising a fluid port and an ultrasound imaging transducer;
activating the ultrasound imaging transducer to obtain a representation of material, optionally tissue and/or fluid injectate delivered from the catheter, external to the internal surface of the lumen; and
injecting fluid out of the fluid port at at least 50 m/sec, piercing through the lumen wall, and into tissue adjacent the lumen wall.

2. The method of claim 1, wherein activating the ultrasound imaging transducer occurs at least during one of the following times: before injecting fluid, during fluid injection, and after fluid injection.

3. The method of claim 2, further comprising determining fluid delivery parameters, such as a fluid pressure waveform, and injecting fluid with the determined fluid delivery parameters, optionally wherein determining fluid delivery parameters is based on an assessment of the obtained representation of tissue.

4. The method of claim 2, further comprising assessing the representation of material, either during fluid delivery or after fluid delivery has stopped, and optionally further comprising injecting additional fluid based on the assessed representation of fluid.

5. The method of claim 4 wherein injecting additional fluid can have the same delivery parameters of different delivery parameters as the first fluid injection.

6. The method of claim 4 wherein assessing the representation is performed automatically with a controller unit via a feedback loop, the controller unit adapted to automatically control the additional fluid delivery.

7. The method of claim 4 wherein assessing the representation comprises assessing a change in the representation compared to representation prior to fluid delivery.

8. The method of claim 4 wherein assessing the representation comprises assessing at least one of a depth of fluid injected and a volume of tissue interacting with the fluid injected.

9. The method of claim 3, wherein the fluid is delivered in pulses, optionally wherein the pulses having varying shapes.

10. The method of claim 1, wherein the representation includes features indicating a boundary between first and second materials, and wherein optionally the representation is a 2-D ultrasound image.

11. The method of claim 1, further comprising includes a contrast agent.

12. The method of claim 1, further comprising determining that the catheter is positioned in an acceptable location within the lumen based on an assessment of the representation.

13. (canceled)

14. A fluid delivery catheter with an ultrasound imaging transducer, comprising:
an elongate member comprising a fluid delivery lumen therein, the elongate member including a fluid exit port in communication with the fluid delivery lumen, wherein the fluid port is sized to create a high velocity fluid jet of at least 50 m/sec at a pressure of at least 750 psi, and the elongate member is adapted to sustain a pressure of at least 750 psi; and
an imaging ultrasound transducer, optionally an array, secured with respect to the elongate member.

15. The fluid delivery catheter of claim 14 wherein the ultrasound transducer is mounted, optionally directly, to the elongate shaft.

16. The fluid delivery catheter of claim 14, further comprising an expandable member secured to the elongate member, wherein optionally the ultrasound imaging transducer is disposed within the expandable member.

17. The fluid delivery catheter of claim 14, wherein the exit port is axially set back away from an outer surface of the elongate member.

18. A fluid delivery system with ultrasonic imaging, comprising:
a fluid catheter comprising: a fluid lumen in communication with a fluid exit port, and an imaging ultrasound transducer, the fluid port sized to create a high velocity fluid jet of at least 50 msec at a pressure of at least 750 psi, wherein the fluid catheter is adapted to sustain a pressure of at least 750 psi;
a controller adapted to receive as input information obtained using the ultrasound transducer, the information indicative of material proximate to the imaging ultrasound transducer, the controller further configured to control at least one aspect of fluid delivery out of the exit port based on the information obtained.

19. The method of claim 1, wherein the injection step comprises, after piercing through the lumen wall, spreading fluid into the tissue.

20-26. (canceled)
